(19) United States
(12) Patent Application Publication
(10) Pub. No.: US 2020/0019995 A1
(43) Pub. Date: Jan. 16, 2020

(54) SYSTEM AND METHOD FOR TARGETING AUDIENCES FOR HEALTH BEHAVIOR MODIFICATION USING DIGITAL ADVERTISEMENTS

(71) Applicants: Mahesh Krishnan, McLean, VA (US); Akshay Krishnan, McLean, VA (US); Anjali Krishnan, McLean, VA (US)

(72) Inventors: Mahesh Krishnan, McLean, VA (US); Akshay Krishnan, McLean, VA (US); Anjali Krishnan, McLean, VA (US)

(21) Appl. No.: 16/032,072

(22) Filed: Jul. 11, 2018

(51) Int. Cl. G06Q 30/02; G06F 17/30; G06N 5/02; G16H 10/60

(52) U.S. Cl. CPC G06Q 30/0271; G06F 17/30864; G06Q 30/0243; G16H 10/60; G06N 5/02

(57) ABSTRACT

The present invention uses micro and nano segmentation to create targeted digital content, in the form of segment specific static ads, pictures, carousel, mobile new feeds, video, canvas and other ad types. For example, if the motivation for a health change in an individual is for the sake of their family, specific images or video from social media may be effective in tying the rationale for the change to the messaging. This content can then be delivered is static ads, in carousels or other media options. The content is targeted to a specific user using conventional and unconventional social media targeting systems to assist with the creation of custom campaign delivered over social media for the purposes of influencing positive health behavior. Data such as including browsing history, social media posts, Interests, Gender, Relationship Status, Educational Status, Age, Location, Language as well as user specific medical and non medical data sources including but not limited to demographics, medical history, treatments, credit score data and report information etc. will be used with a machine learning algorithm to create digital patient phenotypes or cohorts and associate them with the probability that a given digital media campaign will be maximally affect to influence individuals in that cohort to affect the desired behavior change. Example may include what graphic elements are included in the digital materials, the frequency of delivering that content, the channels use etc.

### BACKGROUND

Internet Marketers have successfully used digital and social media platforms to influence individual purchasing and other decisions. This has now extended to the use of micro and nano targeted ads (targeting very small segments of individuals or one individual) used to drive consumer behavior towards purchasing a particular good or service or other related application.

The emerging field of digital therapeutics uses stand-alone digital applications to deliver similar content, with the intent of having the same impact on health outcomes as pharmaceuticals. Examples here include Virta and Omada. One barrier to entry to using such products is both the initial need to download an application to your pc or mobile device, and then to use the new application on a daily basis.

However, large social media platforms such as Facebook, Instagram, Snapchat and others overcome this challenge by offering embedded content in the form of advertising as part of their platform accessed by millions of users at least once and often many times a day.

So as to reduce the complexity and length of the Detailed Specification, and to fully establish the state of the art in certain areas of technology, Applicants herein expressly incorporate by reference all the following materials identified in the paragraph below.

“The Surprising power of Online Experiments”, Harvard Business Review, Ron Kohavi and Stefan Thomke, September-October 2017 issue, https://hbr.org/2017/09/the-surprising-power-of-online-experiments

Applicants believe that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(14)-(3), Applicants will amend the specification to expressly recite the essential material that is incorporated by reference as allowed by the applicable rules.

### SUMMARY OF INVENTION

However, the use of these micro and nano targeted behavioral advertising campaigns for use to positively influence consumer behavior towards improved healthcare related outcomes has not been described. By combining traditional social media based indicators with healthcare specific data, customized behavioral modification campaigns can be created with targeted content meant to, optimize the chances of success of a given behavioral change. Thus resulting in a passive delivery of a behavioral campaign every time the patient encounters an ad on the internet or in a social media platform. Example of such changes may include but are not limited to exercise regimens, weight loss, smoking cessation, health nutrition, or medication adherence.

The present invention uses micro and nano segmentation to create targeted digital content in the form of segment specific static ads, pictures, carousel, mobile new feeds, video, canvas and other ad types. For example, if the motivation for a health change in an individual is for the sake of their family, specific images or video from social media may be effective in tying the rationale for the change to the messaging. This content can then be delivered via static ads, in carousels or other media options. The content is targeted to a specific user using conventional and unconventional social media targeting systems to assist with the creation of custom campaign delivered over social media for the purposes of influencing positive health behavior. Data such as including browsing history, social media posts, Interests, Gender, Relationship Status, Educational Status, Age, Location, Language as well, as riser specific medical and non medical data sources including but not limited to demographics, medical history, treatments, credit score data and report information etc. will be used with a machine learning algorithm to create digital patient phenotypes or cohorts and associate them with the probability that a given digital media campaign will be maximally affect to influence individuals in that cohort to affect the desired behavior change. Examples may include what graphic elements are included in the digital materials, the frequency of delivering that content, the channels used etc.

The present invention also uses digital media platforms to conduct A/B testing for a delivered content to given cohort, and then leverages the variables of click through rates, engagement, for continuous feedback through a machine learning platform to continuously optimize the digital health marketing campaign for those patients in the cohort.

The present invention creates custom audiences for the purposes of disease management. The types of disease management include but are not limited to: behavior modification with regards to diet, exercise, smoking cessation, and medication adherence. Given the behavioral inputs that will positively influence this change vary by individual patient circumstance and social situation, the above mentioned data will be used in a continuous feedback loop to improve micro (the patient cohorts mentioned above) and nano (at an individual patient level) targeting of the digital content delivered to those specific users via social media. For example, for weight loss of exercise, family images could be used to motivate the user to initiate or maintain a diet or smoking cessation campaign.

All of the above features of the present invention will be in compliance with the contemporary and legacy versions of the Health insurance Portability and Accountability Act (HIPAA), including but not limited to the ability of targeted individuals to opt in to a given health related behavior campaign. Aspects of disease management currently authorized under HIPAA would also be a feature.

The present invention further includes the ability to monitor cohorts of patients and users to determine the efficacy of various disease management campaigns by associating the delivery and interaction with the campaign with a desired health outcome. For example, associating the interaction between the digital application and weight loss for a given cohort.

The present invention will be able to be “prescribed” by a healthcare professional, insurance plan, or via self referral by the patient in the same way medications are prescribed today.

### BRIEF DESCRIPTION OF DRAWINGS

FIG. 1 illustrates a system including one embodiment of the process by which the present invention functions and accomplishes its goal of micro or nano targeting an audience utilizing digital health advertisements.

FIG. 2 illustrates a system including one embodiment of the process by which the present invention functions and accomplishes its goal of micro or nano targeting an audience utilizing digital health advertisements.

FIG. 3 depicts a flow chart of the process by which one embodiment of the present invention would function. In one initial path, a consumer desires a health care change, in another initial path, a payer or provider decides on the health care change.

FIG. 4 depicts almost the same flow chart as FIG. 3. However, the one difference is that this version is internationalized, such that it is not restricted to a HIPAA compliant health campaign.

FIG. 5 depicts almost the same flow chart as FIG. 4. However, the difference is it analyzes metrics through the use of machine learning.

### DETAILED DESCRIPTION

The present invention uses micro and nano targeted digital advertisements in the form of static ads, picture, carousel, mobile new feeds, video, canvas and other ad types. This includes the use of social media targeting systems including browsing history, social media posts, Interests, Gender, Relationship Status, Educational Status, Age, Location, and Language. Feedback from the user's engagement with the content will be fed through a machine learning algorithm to allow for continuous refinement of the targeting and content delivery to maximize the chances of the desired behavioral change.

Microtargeting is a marketing strategy that uses consumer data and demographics to identify the interests of specific individuals, and influence their thoughts and actions. Nanotargeting takes this even further, with even more specific data and narrower targets in terms of interests and influence.

In one embodiment, the invention includes a list of patients provided from health records. Alternatively, the invention includes a list created from a prescribable digital therapeutics format, which is when a medical professional provides a patient about information they need after the leave the doctor's office, and then the patient provides their social media ID or phone number. This leads to the creation of a list of patients with additional relevant data.

The invention uses either list in conjunction with existing data aggregation for consumer data and social media to more finely tune advertisements to a limited audience, i.e. micro or nano target the digital advertisements. This tuning would be done via a machine learning platform with the goal of maximizing the effectiveness of a lifestyle change. The machine learning platform uses ongoing social content posted by the user along with click through rates and measures of user engagement to refine the delivered content and frequency of the campaign for maximum effectiveness. Some potential lifestyle changes include, but are not limited to: diet changes, exercise, and medication adherence.

In terms of social media, the present invention is will be deployed over the users internet activities including web searches and social media platforms. Web platform s may include Google ads or similar platform. Some current social media platforms that the invention can, be utilized on include, but are not limited to: Facebook, Instagram, Whatsapp, and Snapchat. As mentioned above, the use of machine learning algorithms assist in the optimization of channel selection, frequency and delivered digital content.

Patients may opt out of the advertisements anytime, either by email, secure messaging or text or on the social media platform directly.

The present invention is applicable in any language, and can utilize modern automated translation techniques, such as Google translate, in order to effectively communicate in multiple languages, and further fine tune advertisements to a micro or nano targeted audience.

Furthermore, the present invention uses medical acid non-medical data sources for the purpose of micro and nano targeting via a machine learning platform. One source is demographic information, such as standard social media demographic targeting categories. Another source is credit score data and report information, including data from databases such as Experian, Equifax and non-financial data, aggregators. The present invention uses the associated data points and changes in the data it receives, such as changes in marital history or changes in home ownership or renting. This data is useful in order to more precisely fine tune the type of ad that a certain segment of the population will he responsive to, in terms of a health-focused digital advertisement.

The present invention also uses digital media platforms to conduct A/B testing, to determine which digital messaging variants work best for any given type of consumer continuously refined by a machine learning platform. The method of All testing analyzes different segments of the online population through views, click-throughs and click-to-action, as well as other standard A/B testing techniques. For efficacy testing, the present invention evaluates the efficacy of the digital therapeutic intervention delivered over social media (including chatbots) compared to a matched virtual control group.

The present invention allows for the consent of the patient to be enrolled in a given behavioral health campaign, no different than a patient agreeing to fill a doctor's prescription and then take the drug as prescribed. The types of behavioral interventions include but are not limited. to: behavior modification with regards to diet, exercise, smoking cessation, and medication adherence.

All of the above features of the present invention will be in compliance with the Health insurance Portability and Accountability Act (HIPAA). The present invention accomplishes this by asking for patient consent for marketing, and specifically for behavior modification and disease management.

The present invention would also cover public service announcements made over social media.

The present invention further includes the ability to monitor cohorts of patients and users to determine the efficacy of various disease management campaigns. One way in which the present invention accomplishes this, is through social media, email and/or electronic questionnaires and surveys. Another way in which the present invention accomplishes monitoring patients and determining the efficacy of various campaigns is through a linkage between enrolled patients and their lab/clinical data (conducted by the sponsoring organization). Specifically, the hospital or health system could check to see if the enrolled patients had a decrease in weight, or other health based improvement.

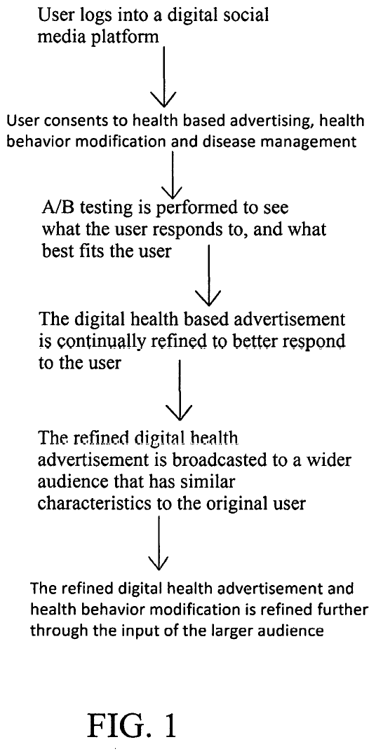

FIG. 1 depicts a flow chart of the process by which one embodiment of the present. invention would function. Step 1: a user logs into a digital social media platform, Step 2: the user consents to health based advertising, health behavior modification and disease management. Step 3: A/B testing is performed to see what the user responds to, and what best fits the user. Step 4: The digital health advertisement is continually refined to better respond to the user. Step 5: The refined digital health advertisements broadcasted to a wider audience that has similar characteristics to the original user. Step 6: The refined digital health advertisement and health behavior modification is refined further through the input of the larger audience.

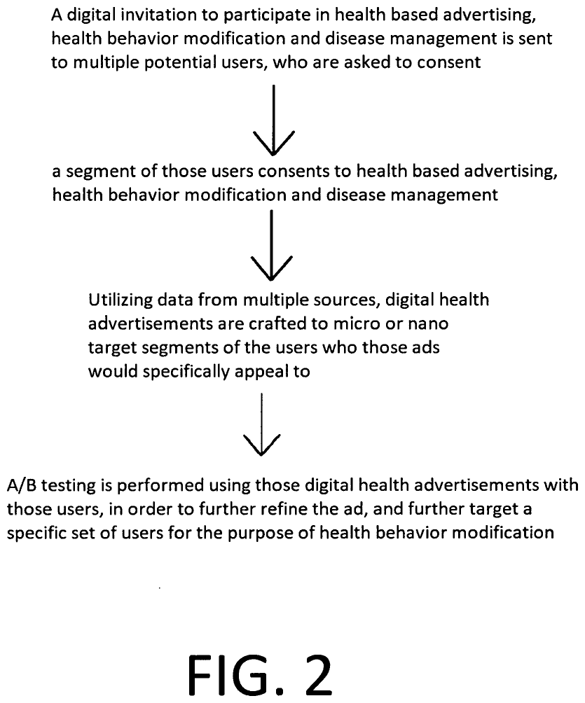

FIG. 2 depicts a flow chart of the process by which one embodiment of the present invention would function, Step 1: digital invitation to participate in health based advertising, health behavior modification and disease management is sent to multiple potential users, who are asked to consent. Step 2: a segment of those users consents to health based advertising, health behavior modification and disease management. Step 3: Utilizing data from multiple sources, digital health advertisements are crafted to micro or nano target segments of the users who those ads would specifically appeal to. Step 4: A/B testing is performed using those digital health advertisements with those users, in order to further refine the ad, and further target a specific set of users for the purpose of health behavior modification.

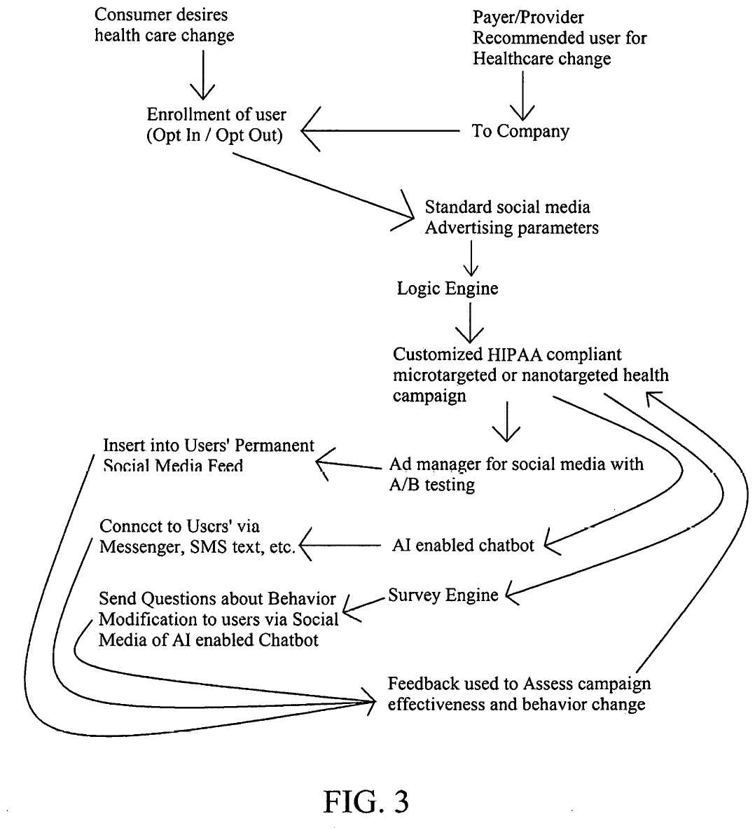

FIG. 3 depicts a flow chart of the process by which one embodiment of the present invention would function. In one initial path, a consumer desires a health care change, and enrolls themselves into the present invention's program, they opt in or opt out. In an alternative initial path, a payer or provider recommends the user for a healthcare change. This message is sent to the company of the user, and then the company enrolls the user into the present invention's program, and the User. Opts in or opts out.

After either initial path, after the user enrolls in the program, the standard social media advertising parameters are determined and customized for that user. This is done through the analysis of a logic engine. The result is a customized HIPAA compliant microtargeted or nanotargeted health campaign. This campaign takes 3 forms, the first is an ad manager for social media, that uses A/B testing to further customize ads for the user. This ad manager is inserted into Users' social media feed, so that the user sees the ads in whichever social media they interact with. The second is an Artificial intelligence enabled chatbot. This chatbot connects to Users via Messenger, SMS text, secured texting or other methods of online chatting. The third is a survey engine, which sends questions about behavior modification to Users via social media. These questions can be sent through the Artificial intelligence enabled chatbot, or they can be sent separately.

All 3 forms of the campaign send feedback that is used to assess campaign effectiveness and behavior change in the user. This feedback is used to make additional changes to the customized HIPAA compliant microtargeted or nanotargeted health campaign, which will result in an even more personalized experience for the User.

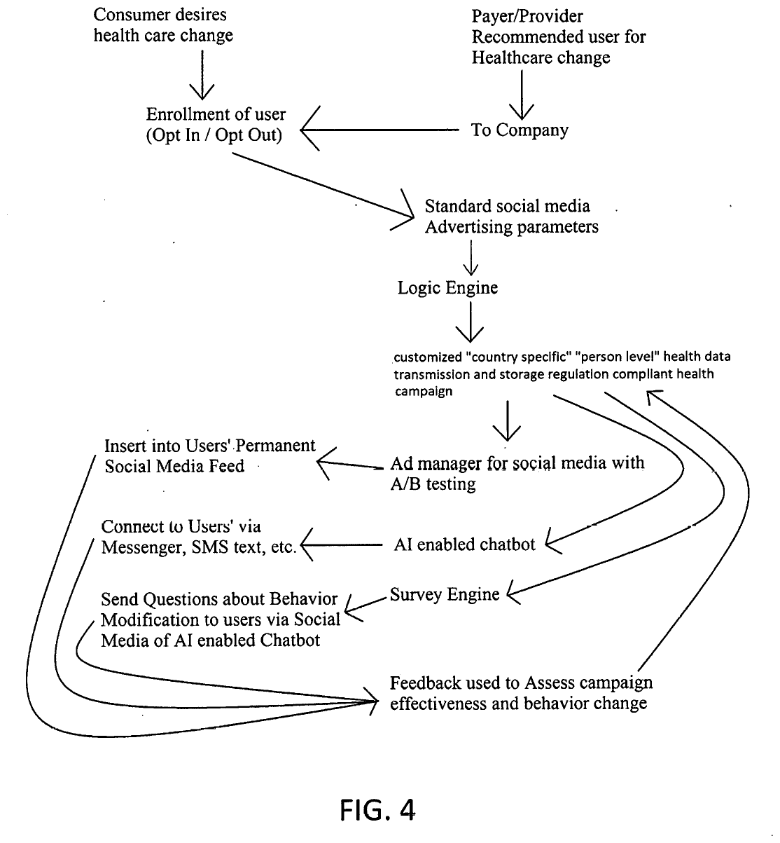

FIG. 4 depicts a flow chart of the process by which one embodiment of the present invention would function. FIG. 4 depicts almost the same flow chart as FIG. 3, However, the one difference is that this version is internationalized, such that it is not restricted to a HIPAA compliant health campaign. Instead, the process creates a customized “country specific” “person level” health data transmission and storage regulation compliant health campaign.

In another embodiment of the present invention, population health management is utilized. Population health management is the aggregation of patient data across multiple health information technology resources, the analysis of that data into a single, actionable patient record, and the actions through which care providers can improve both clinical and financial outcomes. The present invention utilizes population health management by keeping track of the ad campaigns, recording that information, and bringing the analysis of the data gained through the feedback from those campaigns into a single patient record.

In another embodiment of the present invention, the friends of the user on social media can be utilized to help reinforce the different forms of the campaign. It's possible that certain health recommendations could be shared with certain friends of the user on social media, if the user has allowed such sharing. In that situation, the friends on social media would act in a synchronized manner with the campaign, perhaps increasing the chances of a health care change in the user.

In another embodiment of the present invention, the artificial intelligence enabled chatbot would leverage the data from standard social media advertising parameters and other data sources to create a “coach” to help the user achieve the desired health goal. This action would be initially prompted from the first set of data. Subsequent iterations could come from updated data and feedback from the user as they respond the nanotargeted social media add campaign. The artificial intelligence enabled chatbot would ask some standard questions about medication adherence, diet and exercise as well as custom generated responses related to specific social media advertising parameters and responsiveness to the advertising campaign. As such, the feedback loop would continue to change the responses provided by the artificial intelligence enabled chatbot.

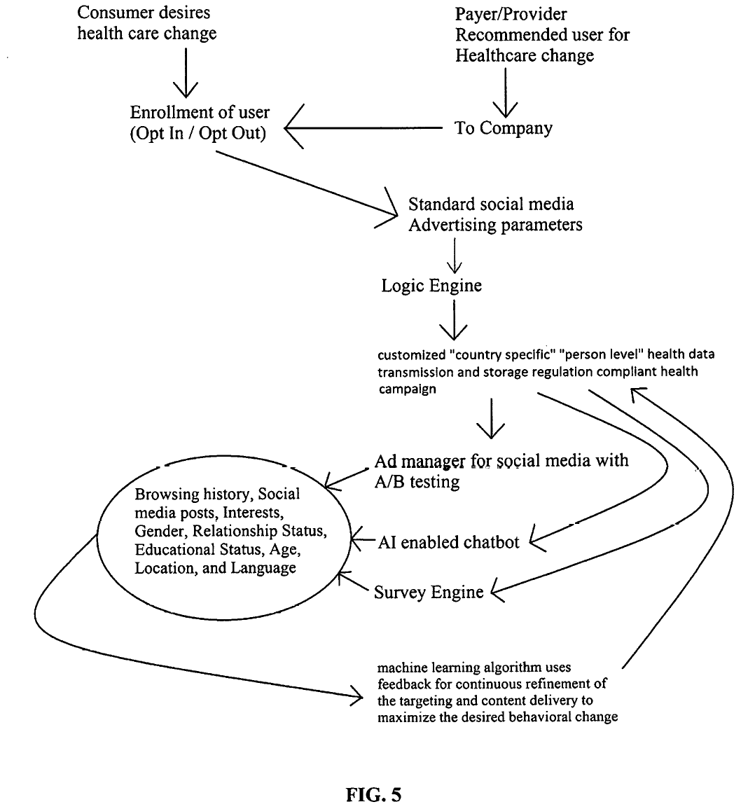

FIG. 5 depicts a flow chart of the process by which one embodiment of the present invention would function. FIG. 5 depicts almost the same flow chart as FIG. 4. However, the difference is that this version analyzes the browsing history, social media posts, Interests, Gender, Relationship Status, Educational Status, Age, Location, and Language. This content will be ted through a machine learning algorithm to allow for continuous refinement of the targeting and content delivery to maximize the chances of the desired behavioral change in future advertisements.

The above descriptions are merely preferred examples of the present invention, and are limited to this invention. Any modifications, equivalent replacements and improvements made within the spirit and principle of the present invention should be included within the scope of protecting this invention.

What is claimed is:

1. A system for targeting audiences for health behavior modification using digital advertisements, comprising:
a program targeting users who enroll in the program;
the program determining and customizing social media advertising parameters for that user through a logic engine;
the program creating a customized HIPAA compliant microtargeted health campaign that takes 3 different forms:
a. the first form is an advertising manager that uses A/B testing to further customize advertisements for the user;
b. the second form is an artificial intelligence enabled chatbot;
c. the third form is a survey engine, which sends questions about behavior modification to the user via social media.

2. The system of claim 1, further comprising:
The program utilizing the techniques of population health management to aggregate patient data across multiple health information technology resources and compile an analysis of that data into a single, actionable patient record;
the program using this single, actionable patient record to keep track of advertising campaigns, analyze the feedback from those campaigns, and create an increasingly more accurate HIPAA compliant microtargeted health campaign for the user.

3. The system of claim 1, further comprising:
the program utilizing the friends of the user on social media to help reinforce health behavior modification;
the program suggesting information to the friends of the user on social media in a synchronized manner with the campaign, such as at the same time that the campaign is sending out the suggested information.

4. The system of claim 1, further comprising:
the program utilizing micro and nano targeted digital advertisements in the form of static ads, picture, carousel, mobile new feeds, video, canvas and other ad types;
the advertisements being based on social media targeting systems including browsing history, social media posts, interests, Gender, Relationship Status, Educational Status, Age, Location, and Language.

5. The system of claim 1, further comprising:
the program collecting feedback from the user's engagement with the content;
the program feeding that feedback through a machine learning algorithm to allow for continuous refinement of targeting and content delivery of the digital advertisements to maximize the chances of the desired behavioral change.

6. A method for targeting audiences for health behavior modification using digital advertisements, comprising:
a program targeting users who enroll in the program;
the program determining and customizing social media advertising parameters for that user through a logic engine;
the program creating a customized HIPAA compliant microtargeted health campaign that takes 3 different forms:
a. the first form is an advertising manager that uses AM testing to further customize advertisements for the user;
b. the second form is an artificial intelligence enabled chatbot;
c. the third form is a survey engine, which sends questions about behavior modification to the user via social media.

7. The method of claim 6, farther comprising:
the program utilizing the techniques of population health management to aggregate patient data across multiple health information technology resources and compile an analysis of that data into a single, actionable patient record;
the program using this single, actionable patient record to keep track of advertising campaigns, analyze the feedback from those campaigns, and create an increasingly more accurate HIPAA compliant microtargeted health campaign for the user.

8. The method of claim 6, further comprising:
the program utilizing the friends of the user on social media to help reinforce health behavior modification;
the program suggesting information to the friends of the user on social media in a synchronized manner with the campaign, such as at the same time that the campaign is sending out the suggested information.

9. The method of claim 6, further comprising:
the program utilizing micro and nano targeted digital advertisements in the form of static ads, picture, carousel, mobile new feeds, video, canvas and other ad types;
the advertisements being based on social media targeting systems including browsing history, social media posts, Interests, Gender, Relationship Status, Educational Status, Age, Location, and Language;

10. The method of claim 6, further comprising:
the program collecting feedback from the user's engagement with the content;
the program feeding that feedback through a machine learning algorithm to allow for continuous refinement of targeting and content delivery of the digital advertisements to maximize the chances of the desired behavioral change.

11. A method for targeting audiences for health behavior modification using digital advertisements, comprising:
a program targeting users who enroll in the program;
the program determining and customizing social media advertising parameters for that user through a logic engine;
the program creating a customized country-specific individual health data transmission and storage regulation compliant health campaign that takes 3 different forms:
a. the first form is an advertising manager that uses A/B testing to further customize advertisements for the user;
b. the second form is an artificial intelligence enabled chatbot;
c. the third form is a survey engine, which sends questions about behavior modification to the user via social media;
the program utilizing the techniques of population health management to aggregate patient data across multiple health information technology resources and compile an analysis of that data into a single, actionable patient record;
the program using this single, actionable patient record to keep track of advertising campaigns, analyze the feedback from those campaigns, and create an increasingly more accurate HIPAA compliant microtargeted health campaign for the user;
the program utilizing the friends of the user on social media to help reinforce health behavior modification;
the program suggesting information to the friends of the user on social media in a synchronized manner with the campaign, such as at the same time that the campaign is sending out the suggested information;
the program utilizing micro and nano targeted digital advertisements in the form of static ads, picture, carousel, mobile new feeds, video, canvas and other ad types;
the advertisements being based on social media targeting systems including browsing history, social media posts, Interests, Gender, Relationship Status, Educational Status, Age, Location, and Language.

12. The method of claim I 1, further comprising:
the advertising manager that is the first form of the health campaign of the program also includes machine learning to limber customize advertisements for the user;
the program collecting feedback from the user's engagement with the content;
the program feeding that feedback through a machine learning algorithm to allow for continuous refinement of targeting and content delivery of the digital advertisements to maximize the chances of the desired behavioral change.
